(19) United States
(12) Patent Application Publication
(10) Pub. No.: US 2024/0336940 A1
(43) Pub. Date: Oct. 10, 2024

(54) SYSTEMS AND METHODS FOR ON-SITE BIOREMEDIATION OF SCRAP MATERIALS

(71) Applicant: MYCOCYCLE, INC., Bolingbrook, IL (US)

(72) Inventor: Joanne Rodriguez, Bolingbrook, IL (US)

(21) Appl. No.: 18/628,394

(22) Filed: Apr. 5, 2024

Related U.S. Application Data
(60) Provisional application No. 63/495,084, filed on Apr. 8, 2023.

(51) Int. Cl. C12P 1/02; C12M 1/00; C12M 3/00; C12N 1/14

(52) U.S. Cl. CPC C12P 1/02; C12M 23/44; C12M 23/52; C12N 1/14

(57) ABSTRACT

Disclosed and claimed are systems and methods for on-site bioremediation of scrap material using a self-contained receptacle.

### RELATED APPLICATIONS

The present patent application claims the priority benefit of U.S. Provisional Patent Application No. 63/495,084, filed Apr. 8, 2023, the content of which is hereby incorporated by reference in its entirety into this disclosure.

### BACKGROUND

Landfills are at 85% capacity, and the waste industry accounts for 16% of total methane emissions. Mismanaged waste results in 400,000+ deaths each year. A need exists for a natural waste solution to address the current waste and emission challenges.

### BRIEF SUMMARY

The present disclosure relates to a receptacle configured to be a self-contained system for remediating waste or scrap materials using fungal cultures.

One aspect of the present disclosure related to a receptacle for producing a bio-based material, including a plurality of holders configured to store scrap material; a scrap material station configured to process scrap material; a treatment station configured to treat the processed scrap material and produce a bio-based material; and a collection station, wherein bio-based material is collected and/or stored.

In an aspect, the receptacle is mobile. In another aspect, the treatment of the scrap material, the production of the bio-based material, and the collection of the bio-based material occur within the receptacle.

In an aspect, the scrap material and/or the production of the bio-based material do not comprise soil.

In an aspect, the receptacle is scalable. In an aspect, the receptacle is used for bioremediation of scrap material. In an aspect, the receptacle is an engineered ecosystem. In another aspect, the receptacle is climate controlled. In another aspect, the receptacle is a container, a subcontainer, a compartment, or an enclosure.

One aspect of the present disclosure is a method for producing bio-based materials, the method including: providing at least one receptacle according to the disclosure; obtaining scrap material; processing the scrap material; treating the scrap material with a bioremediating composition; and producing a bio-based material from the treated scrap material.

In an aspect, processing the scrap material comprises breaking down and/or sterilizing the scrap material. In an aspect, the scrap material comprises coatings, roof materials, shingles, roll goods, single ply membranes, insulation, acoustic tile, wall board, gypsum, drywall, carpet backing, hard surface flooring, exterior sheathing, tires, rubber, crumb rubber, SBR crumb rubber, roof coverboards, and combinations thereof. In an aspect, the scrap material is a raw material. In an aspect, the scrap material is a waste material.

In an aspect, the scrap material and/or the bioremediating composition do not comprise soil.

In an aspect, the bioremediating composition comprises a growth medium and a fungal culture. In an aspect, the growth medium includes a lignin-containing material, sawdust, paper, cardboard, straw, wheat bran, gypsum, straw, soy, hemp, calcium carbonate, magnesium hydroxide, paper, aluminum trihydrate, bulking agents, fillers, and/or fibers.

In an aspect, the fungal culture includes Pleurotus ostreatus, Pleurotus pulmonarius, Ganoderma lucidum, Trametes versicolor, Pleurotus columbinus, Pleurotus eryngii, Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fames spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and/or Lentinula spp.

In another aspect, the bioremediating composition further comprises a sterilizing agent and/or water.

In an aspect, treating the scrap material comprises inoculating the treated scrap material; hydrating the treated scrap material; exposing the treated scrap material to air; incubating the treated scrap material; storing the treated scrap material; and/or drying the treated scrap material.

In an aspect, the production of the bio-based material takes at least about 3 days, alternatively at least about 5 days, alternatively at least about 1 week, alternatively at least about 2 weeks, alternatively at least about 3 weeks, alternatively at least about 4 weeks, alternatively at least about 5 weeks, alternatively at least about 6 weeks, alternatively at least about 7 weeks. In an aspect, the produced bio-based material is collected. In another aspect, the method is automated.

In an embodiment, the present disclosure describes a method of reducing and/or preventing greenhouse gas emissions, the method including on-site remediation of at least one scrap material, wherein the remediation comprises: treating the scrap material with a bioremediating composition; wherein the bioremediating composition comprises a growth medium and a fungal culture; wherein the fungal culture comprises Pleurotus ostreatus, Pleurotus pulmonarius, Ganoderma lucidum, Trametes versicolor, Pleurotus columbinus, Pleurotus eryngii, Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fames spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and/or Lentinula spp.; producing a bio-based material from the treated scrap material; and, wherein the onsite remediation reduces and/or prevents greenhouse gas emissions.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

Various aspects of the present disclosure will now be described, by way of example only, with reference to the attached Figures, wherein:

FIGS. 1A and 1B are illustrations of a receptacle according to an aspect of this disclosure.

FIGS. 2A and 2B are illustrations of a receptacle according to an aspect of this disclosure.

FIG. 3 is a depiction of a method according to an aspect of this disclosure.

FIG. 4 is a depiction of a method according to an aspect of this disclosure.

### DETAILED DESCRIPTION

One aspect of the disclosure relates to decentralizing the processing of scrap and/or waste materials into new performance additive materials via self-contained receptacles. The climate-controlled solution allows users to divert waste from landfills, WTE, or other burdensome processes and create a renewable resource of value-added byproducts. The process is carried out within the receptacle, which creates a perfect ecosystem for the remediation process and, with gypsum materials specifically, does not require the removal of the paper facer, which is required for all other known recycling applications. The receptacle has the ability to reduce the greenhouse gases related to transporting and disposal of materials and will revolutionize the biological transformation of waste on site and in less time.

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

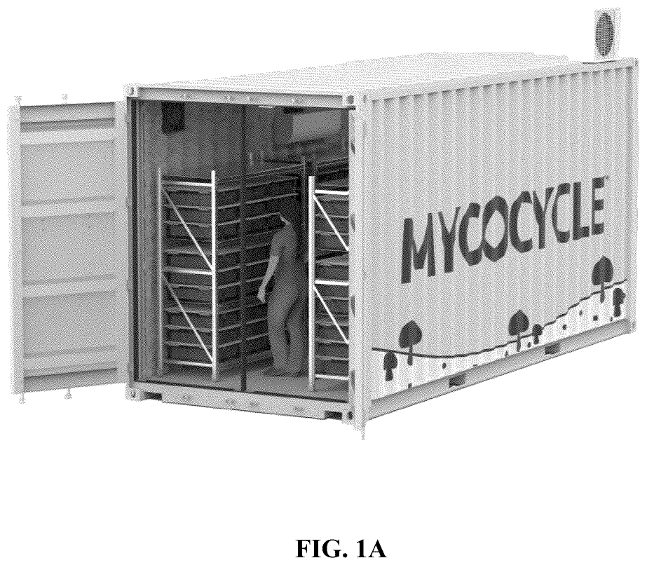

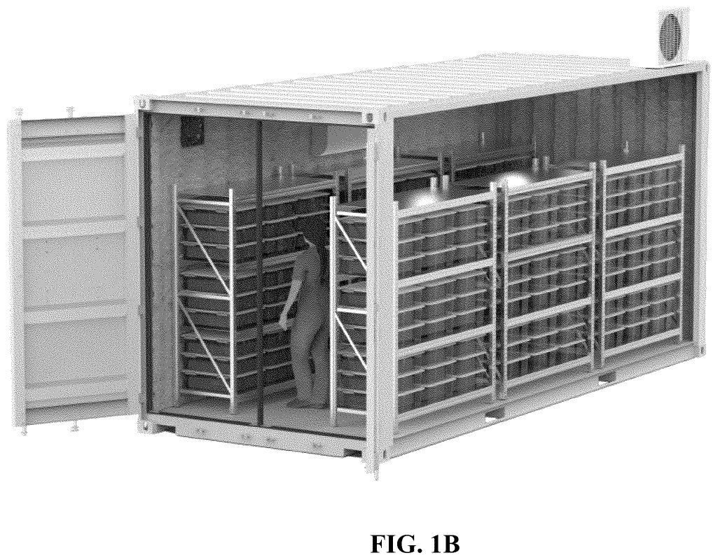

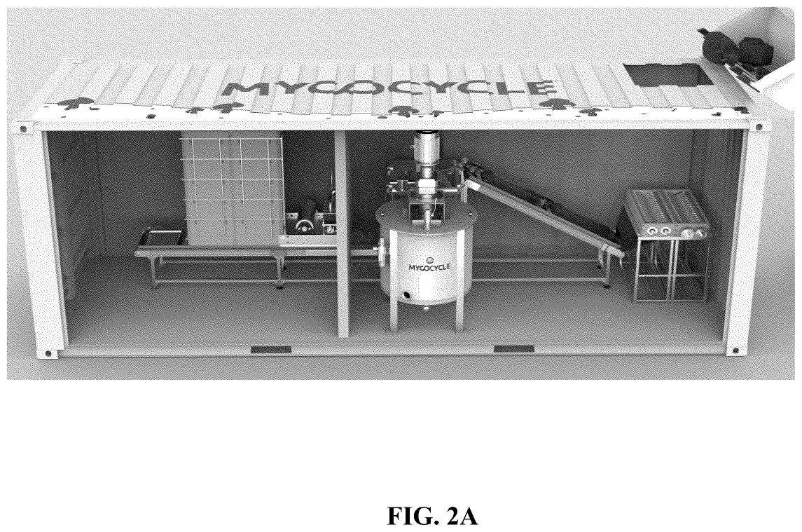

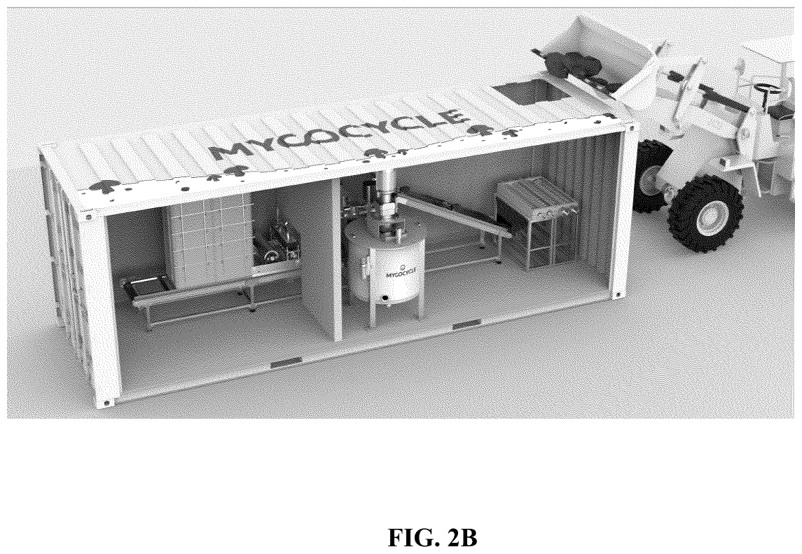

Referring now to FIGS. 1A, 1B, 2A, and 2B, aspects of this disclosure relate to receptacles for producing a bio-based material. A bio-based material is a material that has been synthesized using biotechnological processing. In a non-limiting example, bio-based materials of the present disclosure may be produced using fungal cultures.

The receptacle may be configured to have a plurality of holders configured to store scrap material, a scrap material station configured to process scrap material, a treatment station configured to treat the processed scrap material and produce a bio-based material, and a collection station, wherein bio-based material is collected and/or stored. However, as shown in FIGS. 1A and 1B, the receptacle may only contain a plurality of holders configured to store scrap material and/or a collection station. In this aspect, the receptacle as illustrated in FIGS. 1A and 1B may be used in conjunction with the receptacle of FIGS. 2A and 2B. However, depending on the processing desired, the receptacles as illustrated in FIGS. 1A and 1B and FIGS. 2A and 2B may be used independently of each other.

The receptacle may be used for bioremediation of scrap material. Bioremediation is the use of microorganisms, such as fungal cultures, to consume and/or break down materials, such as scrap materials. Scrap materials may include materials that are left over or unused from a manufacturing process or other processes. This can include raw materials or waste materials, including, but not limited to, construction waste materials. In a non-limiting example, the scrap material may include coatings, roof materials, shingles, roll goods, single ply membranes, insulation, acoustic tile, wall board, gypsum, drywall, carpet backing, hard surface flooring, exterior sheathing, tires, rubber, crumb rubber, SBR crumb rubber, roof coverboards, and combinations thereof.

The receptacle may also be mobile. This is advantageous as it allows the receptacle to be moved to where the scrap material is located rather than requiring the transport of the scrap material to the station.

Another advantage of the disclosed receptacle is that it enables a self-contained environment for the production of bio-based materials. For example, and as shown in FIGS. 2A and 2B, the treatment of the scrap material, the production of the bio-based material, and the collection of the bio-based material occur within the receptacle. The environment in the receptacle can be controlled, and may include further processing equipment such as but not limited to, preprocessing, processing, or postprocessing equipment. Some non-limiting examples include pulverizers, mixers, bioreactors, humidifiers/dehumidifiers, drying ovens, cold or hot presses, form molds, packaging machines, etc. The environment and receptacle may include testing equipment. Some non-limiting examples include spectrophotometry equipment, gas chromatography equipment, mass spectrometry, etc.

The receptacle may be a container, a subcontainer, a compartment, or an enclosure. In some aspects, the receptacle is an engineered ecosystem and/or is climate controlled. An engineered ecosystem may include several sub-containers, sub-compartments, or enclosures, which in turn may be interconnected, partially connected, or completely disconnected. Climate control may include, but is not limited to, humidity controls, temperature controls, airflow controls, pressure controls, water/nitrogen/carbon dioxide controls, and the like.

In non-limiting examples, the receptacle may be an 8 ft container, 10 ft container, 20 ft container, 30 ft container, 40 ft container, or a 40 ft high cube container. In a non-limiting aspect, the receptacle is a 20 ft container. In this aspect, the receptacle is able to process approximately about 10,000 lbs of waste per cycle or approximately about 35,000 lbs of waste per year. This receptacle is also about to process approximately about 53,000 lbs of bi-product per year. In some aspects, the receptacle is also scalable. This allows several receptacles to be used in conjunction to allow for increased production. In non-limiting aspects, at least two receptacles may be used in conjunction, alternatively, at least three receptacles may be used in conjunction, alternatively at least four receptacles may be used in conjunction, or alternatively at least five may be used in conjunction. However, depending on the growth and scale desired, any number of receptacles may be used to achieve the desired scalability.

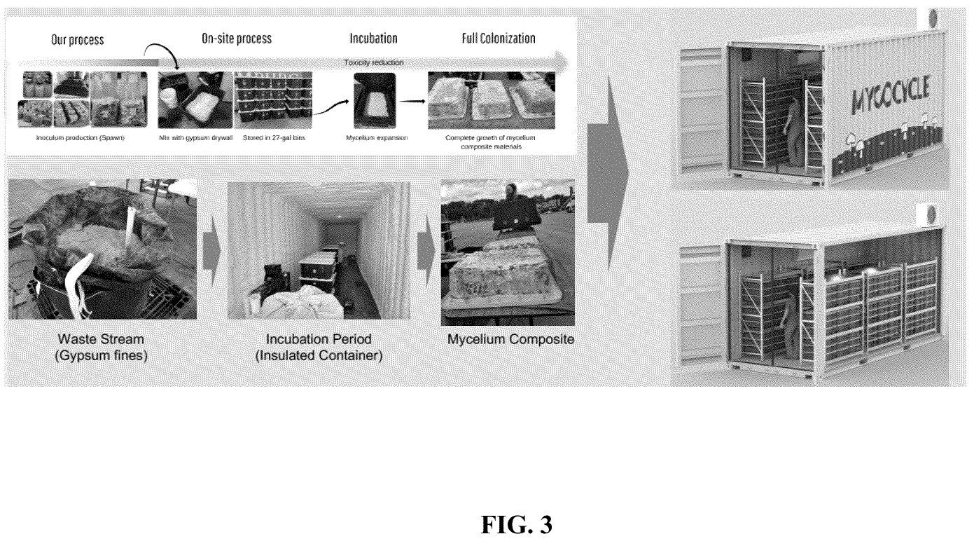

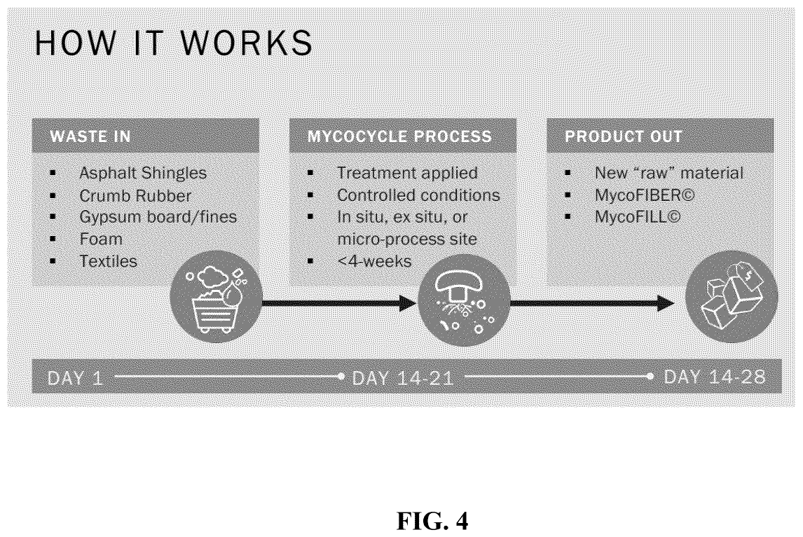

Referring to FIGS. 3 and 4, in some aspects, the receptacle is used in a method for producing bio-based materials. The uniqueness of the receptacle allows, in some aspects, for the automation of this method. In non-limiting examples, this method includes obtaining scrap material, processing the scrap material, treating the scrap material with a bioremediating composition, and producing a bio-based material from the treated scrap material. Processing the scrap material may be done using any conventional techniques known in the art. Non-limiting examples include breaking down and/or sterilizing the scrap material.

To aid in the production of a bio-based material, the processed scrap material must be treated, including treatment with a bioremediating composition. Suitable bioremediating compositions and methods are disclosed in WO Publication 2020/055706, which is incorporated by reference into this disclosure. The bioremediating composition may include a growth medium and a fungal culture. The bioremediating composition may further include a sterilizing agent and/or water. Depending on the scrap material being processed, various growth mediums and fungal cultures may be used. The growth medium may be any suitable structure to promote the growth of the fungal culture. In non-limiting examples, the growth medium may include a lignin-containing material, sawdust, paper, cardboard, straw, wheat bran, gypsum, straw, soy, hemp, calcium carbonate, magnesium hydroxide, paper, aluminum trihydrate, bulking agents, fillers, and/or fibers.

The fungal culture used may be any suitable fungal culture known in the art. Non-limiting examples include Pleurotus ostreatus, Pleurotus pulmonarius, Ganoderma lucidum, Trametes versicolor, Pleurotus columbinus, Pleurotus eryngii, Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fames spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and/or Lentinula spp.

Treating the scrap material may include a variety of steps. These steps may be performed in any suitable order or may be omitted from the method. In addition to treating the scrap material with a bioremediating composition, the scrap material may also be inoculated, hydrated, exposed to air, incubated, stored, and/or dried.

Production of the bio-based material may take at least about 3 days, alternatively at least about 5 days, alternatively at least about 1 week, alternatively at least about 2 weeks, alternatively at least about 3 weeks, alternatively at least about 4 weeks, alternatively at least about 5 weeks, alternatively at least about 6 weeks, alternatively at least about 7 weeks.

In a non-limiting example, the method includes:
a. providing a mobile receptacle, wherein the internal environment of the mobile receptacle is controlled and isolatable, processing the scrap material by breaking it down and sterilizing it, treating the processed scrap material by
b. introducing a bioremediating composition to produce a scrap material-bioremediating mixture;
c. inoculating the mixture, to produce an inoculated mixture;
d. hydrating the inoculated mixture, to produce a hydrated mixture;
e. exposing the hydrated mixture to air, to produce an exposed mixture;
f. incubating the exposed mixture, to produce an incubated mixture;
g. storing and/or drying the incubated mixture, to produce a bio-based material; and
h. collecting the bio-based material.

In non-limiting examples, steps d, e, and/or f may be omitted from the treatment step.

One aspect of the disclosure includes a method of reducing and/or preventing greenhouse gas emissions through on-site remediation. In conventional methods, materials, such as building materials, that can't be reused on-site are transported to a landfill or recycling facility. These materials can also contain contaminants and the disposal process may be challenging. According to the US Environmental Protection Agency, transportation generates the largest share of greenhouse gas emissions (28% of 2021 greenhouse gas emissions) https://www.epa.gov/ghgemissions/sources-greenhouse-gas-emissions. By allowing for on-site remediation, for example using a mobile, isolated, contained environment, the inventors have unexpectantly discovered a method for reducing greenhouse gas emissions while also reducing contaminants. In addition, reuse of the building materials saves the carbon costs of manufacturing.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

### EXAMPLES

### Example 1

In a refitted mobile shipping container, the process of screening and scale up can be performed from examples in PCT/US2019/050128. Organic hulled millet can be hydrated to approximately 60% saturation, and then are sterilized at 250° F. (about 121° C.) for one hour. After cooling, the millet grains can be inoculated with Pleurotus ostreatus (PO, Pearl Oyster) or Pleurotus pulmonarius (PP, Phoenix Oyster) using standard aseptic cultivation techniques. The inoculated millet grains are then incubated for three weeks until fully colonized by mycelium.

Then following two asphalt-containing substrates can be prepared:

| Substrate Component | Substrate 1 | Substrate 2 |
|---|---|---|
| Sawdust mixture* | 50% v/v | 75% v/v |
| Shingle chips** | 25% v/v | 25% v/v |
| Shredded wheat straw** | 25% v/v | 0% v/v |

*Sawdust mixture includes 75% alder sawdust, 15% wheat bran, 10% gypsum, and is pre-hydrated to ~65% saturation with water.
**Pre-hydrated by soaking for 1 hour in filtered water.

For screening, about 8 ounces (about 235 mL) of each Substrate is added to each of four 8-ounce glass jars which are then sterilized at 250° F. (about 121° C.) for one hour. After cooling to ambient temperature, each jar is inoculated with the inoculated millet grains at an inoculation rate of about 5%. Each jar is covered with a lid that included an air filter to prevent influx of foreign microbes.

After incubation at 70° F. (about 21° C.) for one week, abundant fungal growth is observed in each jar. Fungal tissue growth is observed to determine which is most abundant in Pearl Oyster (PO) and Phoenix Oyster (PP), followed by Ganoderma lucidum (GL), Turkey Tail (TV), Blue Oyster (PC), and King Oyster (PE).

### Example 2

In a refitted mobile shipping container, organic hulled millet is hydrated to about 60% saturation and sterilized at 250° F. (about 121° C.) for one hour. After cooling, the millet grains are separated into six batches and each batch was inoculated with one of the following fungal species:
A) Pleurotus ostreatus (PO, Pearl Oyster)
B) Pleurotus pulmonarius (PP, Phoenix Oyster)
C) Pleurotus columbinus (PC, Blue Oyster)
D) Pleurotus eryngii (PE, King Oyster)
E) Ganoderma lucidum (GL, Reishi)
F) Trametes versicolor (TV, Turkey Tail)

Each batch is then incubated for three weeks until fully colonized by mycelium. The following three substrates are prepared:

| | | | Substrate 3 |
|---|---|---|---|
| | | | (no asphalt |
| Substrate Component | Substrate 1 | Substrate 2 | control) |
| Sawdust mixture* | 50% v/v | 25% v/v | 75% v/v |
| Shingle chips** | 25% v/v | 50% v/v | 0% v/v |
| Shredded wheat straw** | 25% v/v | 25% v/v | 25% v/v |

*Sawdust mixture includes 75% alder sawdust, 15% wheat bran, 10% gypsum, and is pre-hydrated to −65% saturation with water.
**Pre-hydrated by soaking for 1 hour in filtered water.

Eighteen 8-ounce glass jars are filled with about 8 ounces (about 235 mL) of each Substrate. Eighteen jars of each Substrate are sterilized at 250° F. for one hour; eighteen jars of each Substrate are sterilized by steam bath pasteurization; and eighteen jars of each Substrate are not sterilized.

Once all jars reach ambient temperature, each of the six inoculated millet grain batches are added to nine jars at an inoculation rate of about 5% using standard aseptic cultivation techniques as follows:
1) Substrate 1, sterilized to 250° F. for one hour via autoclave
2) Substrate 1, pasteurized by 160° F. water bath
3) Substrate 1, not sterilized
4) Substrate 2, sterilized to 250° F. for one hour via autoclave
5) Substrate 2, pasteurized by 160° F. water bath
6) Substrate 2, not sterilized
7) Substrate 3, sterilized to 250° F. for one hour via autoclave
8) Substrate 3, pasteurized by 160° F. water bath
9) Substrate 3, not sterilized

The inoculated substrate jars are incubated at 70° F. (about 21° C.) for one week. Fungal growth is assessed each day as a function of the degree of colonization observed. Samples are collected for analytical testing at mix-up of the three mixes, when each mix of species, substrate, and sterilization treatment reaches about 50% colonization, and again when each jar reached 100% colonization.

### Example 3

In a refitted mobile shipping container, two top-performing fungal species from Example 2 are selected for scale-up testing. Each species are inoculated onto organic hulled millet that is hydrated to about 60% saturation, sterilized at 250° F. (about 121° C.) for one hour, and is cooled to ambient temperature. Six Substrates are prepared as follows:

| Substrate | Substrate | | | | | |
|---|---|---|---|---|---|---|
| Component | 4 | 5 | 6 | 7 | 8 | 9 |
| Sawdust | 0% | 10% | 0% | 10% | 25% | 50% |
| mixture* | v/v | v/v | v/v | v/v | v/v | v/v |
| Shingle | 100% | 90% | 90% | 75% | 50% | 0% |
| chips** | v/v | v/v | v/v | v/v | v/v | v/v |
| Shredded | 0% | 0% | 10% | 15% | 25% | 50% |
| wheat straw** | v/v | v/v | v/v | v/v | v/v | v/v |

*sawdust mixture included 75% alder sawdust, 15% wheat bran, 10% gypsum, and was pre-hydrated to ~65% saturation with water.
**Pre-hydrated by soaking for 1 hour in filtered water.

Twenty-four 5-L containers are filled with each Substrate, for a total of 144 5-L containers. For each Substrate, six containers were sterilized at 250° F. (about 121° C.) for one hour; six are pasteurized using a 160° F. water bath, six are soaked in a strongly alkaline (pH 12) solution, and six are not sterilized.

For each combination of Substrate and sterilization method, three containers are inoculated with each species of incubated grain spawn under aseptic conditions, as follows:

Fungal Species #1—Pearl Oyster (n=72 Containers):

| | 250° F. @ 1 | | Alkaline | No |
|---|---|---|---|---|
| | hour | Pasteurization | Soak | Sterilization |
| Substrate 4 | 3 containers | 3 containers | 3 containers | 3 containers |
| Substrate 5 | 3 containers | 3 containers | 3 containers | 3 containers |
| Substrate 6 | 3 containers | 3 containers | 3 containers | 3 containers |
| Substrate 7 | 3 containers | 3 containers | 3 containers | 3 containers |
| Substrate 8 | 3 containers | 3 containers | 3 containers | 3 containers |
| Substrate 9 | 3 containers | 3 containers | 3 containers | 3 containers |

Fungal Species #2—Turkey Tail (n=72 Containers):

| | 250° F. @ 1 | | Alkaline | No |
|---|---|---|---|---|
| | hour | Pasteurization | Soak | Sterilization |
| Substrate 4 | 3 containers | 3 containers | 3 containers | 3 containers |
| Substrate 5 | 3 containers | 3 containers | 3 containers | 3 containers |
| Substrate 6 | 3 containers | 3 containers | 3 containers | 3 containers |
| Substrate 7 | 3 containers | 3 containers | 3 containers | 3 containers |
| Substrate 8 | 3 containers | 3 containers | 3 containers | 3 containers |
| Substrate 9 | 3 containers | 3 containers | 3 containers | 3 containers |

After inoculation, each container is incubated at 70° F. (about 21° C.) for three weeks, with fungal growth progression is observed and should be documented daily as a function of degree of colonization. Samples are collected for analytical testing at mix-up of the three mixes, when each mix of species, substrate, and sterilization treatment reaches about 50% colonization, and again when each jar reaches 100% colonization. Collected data is analyzed using analysis of variance (ANOVA) with repeated measures on the second factor. A Fisher's LSD analysis is performed for any significant effects. The statistical significance level is set at p<0.05. Statistical analysis is performed using SPSS software and data is interpreted hierarchically.

From the different fungi, the fungi with greatest growth rates can be selected and colonization of this combination with the selection can be monitored until colonization reaches 50% and 100%.

### Example 4

In a refitted mobile shipping container, a controlled, full, multi-factorial mesocosm project at the field scale can be set up with the following three factors: Mushroom Spawn in the form of mycelium; New/discarded material (gypsum drywall); and Inoculation ratios (% of spawn per material total).

### Methods

Recovered gypsum drywall waste is supplied and processed on-site over the course of four weeks. The recovered gypsum is treated with a mushroom spawn (inoculum). The treated material is placed in 27-gal containers (50×) and supersacks (4×). In this example a total of 3,250 lbs. of treated material is placed in incubation conditions for monitoring.

### Mycelium Growth

Treatment and environmental conditions must be adequate for healthy growth and, therefore, mycological conversion of the waste material into new mycelium-based composites. A percentage of total bins under each qualitative category of mycelium growth (Best, Good, Okay) can provide evaluating data.

### Environmental Quality Testing

The base and collected samples are analyzed for various environmental factors such as percent moisture; levels Mercury by method 7471B; Metals (Antimony, Arsenic, Beryllium, Cadmium, Chromium, Copper, Lead, Nickel, Selenium, Silver, Thallium, Zinc) by method 3050B; and VOC by method 8260B.

### Humidity (as Moisture Percentages) by ASTM D2974

One expects humidity to increase with the addition of the mushroom spawn and decrease over time. A steady decrease over time implies that the mycelium consumed the moisture available in the substrate blends reflective of healthy growth throughout the duration of the project.

### Mercury by Method 7471B

Mercury has a high degree of toxicity, so all the values should be below Human Health Screening Levels as presented by the California Office of Environmental Health Hazard Assessment with a value of 18 mg/kg (ppm).

### Priority Pollutant Metals by Method 3050B

The presence and values of 12 metals are measured over time. Zinc is a common metal found not only in gypsum drywall but also in conventional mushroom cultivation substrates such as hardwood sawdust. It would not be surprising if it were the metal with the highest presence in the samples analyzed. Overall, it is not uncommon to observe an increase in the uptake of these metals as the mycelium continues to degrade its substrate via enzymatic/oxidative mechanisms to obtain its nutrition.

### Volatile Organic Compounds by Method 8260B

VOCs are monitored over the duration of the program. Acetone is a very common organic solvent with a rapid evaporation rate and is an excellent degreaser and cleaner for all common metals, glass, porcelain, many plastics, and synthetic fibers. It is one VOC fungi is able to degrade over time.

### Mechanical Testing

Mechanical testing is also done to determine the compressive strength.

The tests can show characteristics similar to low density or foamed materials such as EPS or expanded polystyrene (unicell). There are no standards or norms applicable to the subject of mycelium composites. This opens the door to experimenting with different production processes, such as cold or hot pressing, to increase these composites' density and mechanical properties. It is also important to perform other types of destructive and non-destructive tests to determine the physical properties of mycelium composites, such as acoustic tests, fire resistance tests, permeability, and weathering, among others.

### Example 5—Field-Based Remediation

The method disclosed in Example 4 is used to scale on 2-tons of gypsum drywall waste. However, the variables can be limited to 1-species and 2-species blends. The gypsum materials are mixed with the inoculum and incubated for 2-4 weeks. Quantitative measurements are taken on the initial sample set up, batch test, and post-treatment materials and the results are analyzed.

After the first 30 days in primary incubation, the molded materials with the highest percentage of colonization by the fungus are selected. These samples are then placed in a box for transport to enter post-production process. The secondary incubation process takes place wherein the weight and dimensions of each material are measured and labeled with keys for its identification. It is was then placed in a larger container for a secondary incubation period of 5-7 days.

Drying and finishing: The materials are removed from secondary incubation and exposed to a drying process until approximately 30% of its initial weight is lost. Molds (forms) are used to prepare the mycelium composites at the time of inoculation. The samples are in primary incubation (treatment) for the 30 days that the method is running, plus five days in secondary incubation before drying off for two days

A total of 3 replicates of each treatment for a total of 12 samples are then sent to the lab for mechanical testing.

The complete disclosure of all patents, patent applications, publications, and electronically available material cited herein are incorporated by reference. In the event that any inconsistency exists between the disclosure of the present application and the disclosure(s) of any document incorporated herein by reference, the disclosure of the present application shall govern.

It will be understood that while the invention has been described in conjunction with the detailed description thereof, the foregoing description is intended to illustrate and not limit the scope of the invention, which is defined by the scope of the appended claims. The foregoing detailed description and examples have been given for clarity of understanding only. No unnecessary limitations are to be understood therefrom. The invention is not limited to the exact details shown and described, for variations obvious to one skilled in the art will be included within the invention defined by the claims. Other aspects, advantages, and modifications are within the scope of the following claims.

1. A receptacle for producing a bio-based material, comprising:
a plurality of holders configured to store scrap material;
a scrap material station configured to process scrap material;
a treatment station configured to treat the processed scrap material and produce a bio-based material;
and a collection station, wherein bio-based material is collected and/or stored.

2. The receptacle of claim 1, wherein the station is mobile.

3. The receptacle of claim 1, wherein the treatment of the scrap material, the production of the bio-based material, and the collection of the bio-based material occur within the receptacle.

4. The receptacle of claim 1, wherein the scrap material and/or the production of the bio-based material do not comprise soil.

5. The receptacle of claim 1, wherein the receptacle is scalable.

6. The receptacle of claim 1 wherein the receptacle is used for bioremediation of scrap material.

7. The receptacle of claim 1, wherein the receptacle is an engineered ecosystem, climate controlled, a container, a subcontainer, a compartment, and/or an enclosure.

8. (canceled)

9. (canceled)

10. A method for producing bio-based materials, the method comprising:
providing at least one receptacle according to claim 1;
obtaining scrap material;
processing the scrap material;
treating the scrap material with a bioremediating composition; and
producing a bio-based material from the treated scrap material.

11. The method of claim 10, wherein processing the scrap material comprises breaking down and/or sterilizing the scrap material.

12. The method of claim 10, wherein the scrap material comprises coatings, roof materials, shingles, roll goods, single ply membranes, insulation, acoustic tile, wall board, gypsum, drywall, carpet backing, hard surface flooring, exterior sheathing, tires, rubber, crumb rubber, SBR crumb rubber, roof coverboards, and combinations thereof.

13. (canceled)

14. (canceled)

15. The method of claim 10, wherein the bioremediating composition and/or the scrap material do not comprise soil.

16. The method of claim 10, wherein the bioremediating composition comprises a growth medium and a fungal culture.

17. The method of claim 16, wherein the growth medium comprises a lignin-containing material, sawdust, paper, cardboard, straw, wheat bran, gypsum, straw, soy, hemp, calcium carbonate, magnesium hydroxide, paper, aluminum trihydrate, bulking agents, fillers, and/or fibers.

18. The method of claim 16, wherein the fungal culture comprises Pleurotus ostreatus, Pleurotus pulmonarius, Ganoderma lucidum, Trametes versicolor, Pleurotus columbinus, Pleurotus eryngii, Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fames spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and/or Lentinula spp.

19. The method of claim 10, wherein the bioremediating composition further comprises a sterilizing agent and/or water.

20. The method of claim 10, wherein treating the scrap material further comprises inoculating the scrap material; hydrating the scrap material; exposing the scrap material to air; incubating the scrap material; storing the scrap material; and/or drying the scrap material.

21. The method of claim 10, wherein production of the bio-based material takes at least about 3 days, alternatively at least about 5 days, alternatively at least about 1 week, alternatively at least about 2 weeks, alternatively at least about 3 weeks, alternatively at least about 4 weeks, alternatively at least about 5 weeks, alternatively at least about 6 weeks, alternatively at least about 7 weeks.

22. The method of claim 10, wherein the method is automated and the produced bio-based material is collected.

23. (canceled)

24. The method of claim 10, wherein the method comprises post-processing the produced bio-based material to alter its structural properties.

25. A method of reducing and/or preventing greenhouse gas emissions, the method comprising:
on-site remediation of at least one scrap material, wherein the remediation comprises:
treating the scrap material with a bioremediating composition;
wherein the bioremediating composition comprises a growth medium and a fungal culture;
wherein the fungal culture comprises Pleurotus ostreatus, Pleurotus pulmonarius, Ganoderma lucidum, Trametes versicolor, Pleurotus columbinus, Pleurotus eryngii, Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fames spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and/or Lentinula spp.;
producing a bio-based material from the treated scrap material; and,
wherein the onsite remediation reduces and/or prevents greenhouse gas emissions.
